(19) United States
(12) Patent Application Publication
(10) Pub. No.: US 2005/0214683 A1
(43) Pub. Date: Sep. 29, 2005

(54) Method and apparatus for reforming film and controlling slimming amount thereof

(75) Inventors: Eiichi Nishimura, Nirasaki-shi (JP); Takashi Tanaka, Nirasaki-shi (JP); Gen You, Nirasaki-shi (JP); Minoru Honda, Amagasaki-shi (JP); Mitsuaki Iwashita, Nirasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: TOKYO ELECTRON LIMITED, Tokyo (JP)

(21) Appl. No.: 11/064,088

(22) Filed: Feb. 24, 2005

(30) Foreign Application Priority Data
Feb. 24, 2004 (JP) 2004-047611

(51) Int. Cl. G03C 5/00; A61N 5/00; G21G 5/00

(52) U.S. Cl. 430/296; 430/942; 250/492.3

(57) ABSTRACT

In a film reforming method for reforming a film layer to be reformed by irradiating electron beams thereon, the electron beams are irradiated in a state where the film layer is cooled. Further, in a slimming amount controlling method for controlling a slimming amount of a resist film layer, the slimming amount thereof is controlled by the irradiation amount of electron beams irradiated thereon in a state where the resist film layer having a specified opening dimension is cooled. Furthermore, in a film reforming apparatus including a mounting unit for mounting thereon an object to be processed and an electron beam irradiating unit for irradiating electron beams on the object disposed on the mounting unit to thereby reform a film layer to be reformed, formed on an object, the electron beams are irradiated from the electron beam irradiating unit in a state where the film layer is cooled by a cooling unit provided in the mounting unit.

### FIELD OF THE INVENTION

The present invention relates to a method and an apparatus for reforming a film and controlling a slimming amount thereof; and, more particularly, to a method and an apparatus for reforming a film and controlling a slimming amount thereof, which are capable of suppressing a dimension change in a pattern opening of a resist film layer.

### BACKGROUND OF THE INVENTION

Due to a remarkably fast development of a lithography technique, a wiring structure of a semiconductor device has been rapidly miniaturized and multilayered. In a lithography process, a resist pattern is formed into a specified pattern by exposing a photoresist formed on a film layer to be etched to light and, then, the film layer is etched by using the resist pattern as a mask, thereby forming a wiring pattern. In a current mass production process, a KrF excimer laser (wavelength 248 nm) is being used as an exposure light source and, further, a miniaturization structure in the order of 0.15 μm is being realized. However, in order to meet a design rule of less than 0.15 μm to be required in a near future due to a further miniaturization, the lithography technique using an ArF excimer laser (wavelength 193 nm) or a fluoride dimmer F₂ is currently being developed. If the lithography technique meets the design rule of less than 0.15 μm, there is required a photoresist material capable of suppressing a line edge roughness with a high resolution and a good etching resistance. Accordingly, a development of the photoresist material satisfying such conditions is in active progress.

As for a photoresist material, a photoresist material containing an aromatic ring having a good etching resistance is being used for the KrF excimer laser. Since, however, the aromatic ring has an absorption band around a wavelength of 193 nm, the photoresist material containing an aromatic ring is not usable for the design rule of less than 0.15 μm, wherein the ArF excimer laser is employed. Accordingly, various photoresist materials for the ArF excimer laser, which contain no aromatic ring, are currently being developed. For example, Reference 1 discloses therein a photoresist material combining adamanthyl methacrylate having an etching resistance and copolymer of t-butyl methacrylate. Such photoresist material does not contain a double bond, e.g., an aromatic ring, in an adamanthyl group and thus has sufficient transparency at the wavelength of 193 nm. Moreover, the same kind of photoresist material for the ArF excimer laser is suggested in Reference 2.

However, the ArF photoresist material, which contains no aromatic ring, has an insufficient etching resistance and, further, a side surface of a resist pattern becomes rough during an etching process. As a result, an original resist pattern cannot be precisely transcribed on a film layer to be etched, which may lead to a defect in a circuit or the like. To overcome such a problem, the photoresist film layer is hardened by performing an optical process in which ultraviolet rays or the like are used on the photoresist film layer, so that the etching resistance can be improved. As for a technique for hardening a photoresist film layer through an optical process, techniques disclosed in References 3 and 4 have been known.

Referring to Reference 3, there is provided a photoresist having a resist pattern composed of a first pattern portion having a first width and a second pattern portion having a second width greater than the first width. The technique disclosed therein is used for exclusively hardening the second pattern portion having a greater width than that of the first pattern portion by irradiating a light only on the second pattern portion without irradiating the light on the first pattern portion. When light is irradiated from a light source, temperature of the photoresist is maintained below 90° C. (preferably, a room temperature). Since a larger pattern is more easily subjected to a pattern contraction during an etching process, such technique tends to be used to suppress the pattern contraction during the etching process by way of hardening the second pattern portion that is a large pattern portion. The light from the light source used for the hardening process is ultraviolet rays or electron beams.

Disclosed in Reference 4 is the technique for suppressing a transformation of a resist pattern by irradiating electron beams on an ArF photoresist film layer to harden it. In such case, there is no description about electron beam irradiation conditions. Besides, as for another technique for hardening resin through the irradiation of electron beams, there are provided a method for curing a curable composition and a method for manufacturing a color filter, respectively, disclosed in References 5 and 6.
[Reference 1] FUJITSU. 50, 4. (07, 1999) pp. 253-258
[Reference 2] U.S. Pat. No. 6,749,989
[Reference 3] U.S. Pat. No. 5,648,198
[Reference 4] U.S. Pat. No. 6,569,778
[Reference 5] U.S. Pat. No. 5,789,460
[Reference 6] Japanese Patent Laid-open Application No. 2002-031710

However, in case of the techniques disclosed in References 3 to 6, electron beams are irradiated in a temperature range requiring a heating. Thus, for example, as illustrated in FIGS. 11A and 11B, a photoresist film layer 2 formed on a film layer 1 to be etched becomes contracted due to an irradiation of electron beams or the like from a state shown in FIG. 11A to a state shown in FIG. 11B. Accordingly, a critical dimension (CD) of an opening of a resist pattern 2A changes (extends) and, further, the original resist pattern 2A cannot be precisely transcribed on the film layer to be etched. The pattern contraction is conjectured to be due to a secession of CO gas or the like from the photoresist film layer by an excessive heat (e.g., a reaction heat) generated when irradiating electron beams or the like from the light source. Further, t in FIG. 11B indicates a reduced film thickness.

Furthermore, as for a photoresist material for meeting a multilayered wiring structure, a tri-layer resist, a bi-layer resist and the like have been developed. In such case, a photoresist film layer for forming a resist pattern is formed as an uppermost layer, and a film having an etching resistance is formed as a lower layer thereunder. Thus, the photoresist film layer serves as a mask for the lower layer film, and the lower layer film serves as a mask for etching a film thereunder. Even in such a case, the uppermost photoresist film layer has suffered the aforementioned drawbacks.

### SUMMARY OF THE INVENTION

It is, therefore, an object of the present invention to provide a method and an apparatus for reforming a film, capable of precisely transcribing an original resist pattern on a film layer to be etched by suppressing a contraction of a photoresist film layer in a curing process performed thereon through an irradiation of electron beams and further preventing a defect in a circuit. Further, another object of the present invention is to provide a method for controlling a slimming amount thereof through an irradiation of electron beams.

In accordance with an aspect of the present invention, there is provided a film reforming method for reforming a film layer to be reformed by irradiating electron beams thereon, wherein the electron beams are irradiated in a state wherein the film layer to be reformed is cooled.

In accordance with another aspect of the present invention, there is provided a slimming amount controlling method for controlling a slimming amount of a resist film layer by controlling the irradiation amount of electron beams irradiated thereon in a state wherein the resist film layer having a specified opening dimension is cooled.

In accordance with still another aspect of the invention, there is provided a film reforming apparatus including a mounting unit for mounting thereon an object to be processed and an electron beam irradiating unit for irradiating electron beams on the object disposed on the mounting unit to thereby reform a film layer to be reformed, formed on the object, wherein the electron beams are irradiated from the electron beam irradiating unit in a state wherein the film layer is cooled by a cooling unit provided in the mounting unit.

The present invention can provide a method and an apparatus for reforming a film, which are capable of precisely transcribing an original resist pattern on a film layer to be etched by suppressing a contraction of a photoresist film layer in a curing process performed thereon through an irradiation of electron beams and further preventing a defect in a circuit. Further, the present invention can also provide a method for controlling a slimming amount thereof through an irradiation of electron beams.

### BRIEF DESCRIPTION OF THE DRAWINGS

The above and other objects and features of the present invention will become apparent from the following description of preferred embodiments, given in conjunction with the accompanying drawings, in which:

FIG. 1 is a diagram showing an electron beam processor appropriate for a film reforming method of the present invention;

FIG. 2 illustrates a top view describing an exemplary arrangement of electron beam tubes of the electron beam processor shown in FIG. 1;

FIGS. 3A to 3C respectively provide conceptual diagrams depicting processes of the film reforming method of the present invention;

FIGS. 4A and 4B present graphs showing a result of an EB curing process performed by using the film reforming method of the present invention, wherein FIG. 4A depicts a relationship between an EB cure time and a CD of a resist pattern and FIG. 4B shows a relationship between an EB cure time and a resist film thickness;

FIG. 5 represents a graph illustrating a relationship between an EB cure time and a contraction percentage of a photoresist film layer, which is obtained when an EB curing process is carried out by using the film reforming method of the present invention;

FIG. 6 offers a graph illustrating a relationship between an EB cure time and an etching rate, which is obtained when a film layer to be etched is etched via a photoresist film layer processed by using the film reforming method of the present invention;

FIG. 7 sets forth a graph illustrating a relationship between an EB cure time and a contraction percentage of a photoresist film layer, which is obtained when an EB curing process is carried out by using the film reforming method of the present invention;

FIG. 8 provides a graph depicting a relationship between an EB cure time and a CD of a resist pattern of the photoresist film layer, which is obtained when an EB curing process is carried out by using the film reforming method of the present invention;

FIGS. 9A to 9C present conceptual diagrams describing a process performed by using a slimming amount controlling method of the present invention;

FIGS. 10A to 10E represent conceptual diagrams illustrating a process performed when the film reforming method of the present invention is applied to a tri-layer photoresist film layer; and

FIGS. 11A and 11B offer conceptual diagrams showing a process performed by using a conventional film reforming method.

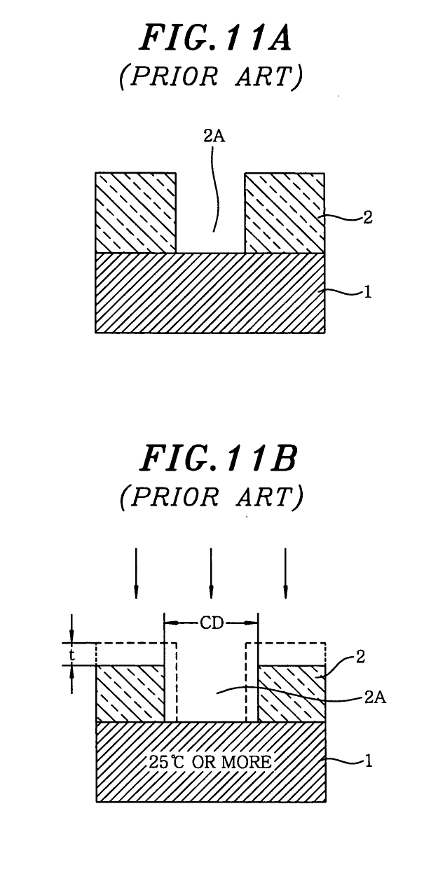

### DETAILED DESCRIPTION OF THE PREFERRED EMBODIMENT

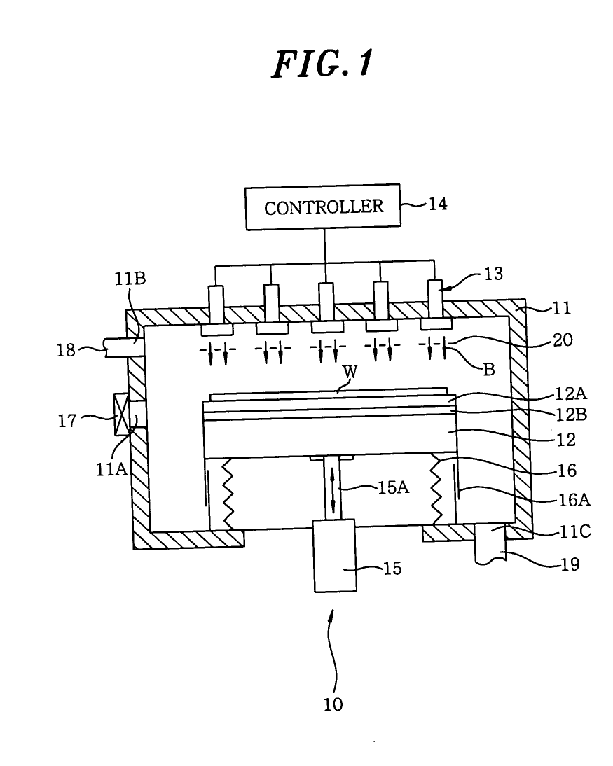

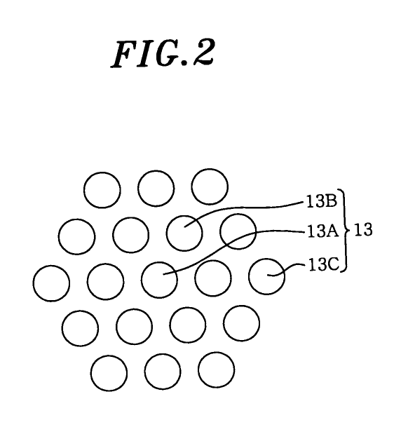

Hereinafter, the present invention will be described based on preferred embodiments shown in FIGS. 1 to 10E. A film reforming method of the present invention employs a film reforming apparatus of the present invention, e.g., an electron beam processor shown in FIGS. 1 and 2. First, the electron beam processor of this embodiment will be described and, then, a film reforming method and a slimming amount controlling method which use the electron beam processor will be described.

As shown in FIG. 1, an electron beam processor 10 of this embodiment includes a depressurizable processing chamber 11 made of aluminum or the like; a mounting table 12 having a cooling unit 12A, positioned at a central bottom surface of the processing chamber 11; a plurality of (e.g., nineteen) electron beam units 13 arranged in a concentric circular shape on a top surface of the processing chamber 11 facing the mounting table 12; and a controller 14 for controlling the mounting table 12, the electron beam units 13 or the like. In a state where a wafer W is cooled by the cooling unit 12A operated under the control of the controller 14, electron beams are irradiated on an entire surface of the wafer W on the mounting table 12 from the electron beam units 13, thereby reforming a photoresist film layer to be described later. Hereinafter, the reforming process is referred to as an EB curing process.

An elevating mechanism 15 is connected to a bottom surface of the mounting table 12, and the mounting table 12 moves up and down via a ball screw 15A of the elevation mechanism 15. The bottom surface of the mounting table 12 and that of the processing chamber 11 are connected by an expansible/contractible bellows 16 made of stainless steel and, further, an inner space of the processing chamber 11 is airtightly maintained by the bellows 16. Moreover, a loading/unloading port 11A of the wafer W is formed at a peripheral surface of the processing chamber 11, and a gate valve 17 is attached to the loading/unloading port 11A in such a way that it can be opened and closed. In addition, a gas supply port 11B is formed above the loading/unloading port 11A of the processing chamber 11, and a gas exhaust port 11C is formed at the bottom surface of the processing chamber 11. Furthermore, a gas supply source (not shown) is connected to the gas supply port 11B via a gas supply pipe 18, and a vacuum exhaust device (not illustrated) is connected to the gas exhaust port 11C via the gas exhaust pipe 19. Besides, a reference numeral 16A in FIG. 1 indicates a bellows cover.

Provided on the top surface of the mounting table 12 is a heater 12B that can be used to heat the wafer W to keep it at a desired temperature if necessary. As illustrated in FIG. 2, the nineteen electron beam units 13 include, e.g., a first electron beam set having a single first electron beam tube 13A positioned at a central top surface of the processing chamber 11; a second electron beam set having six second electron beam tubes 13B arranged around the first electron beam set in an approximately concentric circular shape; and a third electron beam set having twelve third electron beam tubes 13C arranged around the second electron beam set in an approximately concentric circular shape, wherein an output of each set is separately controllable. The first to third electron beam tubes 13A to 13C have electron beam transmitting windows provided exposedly in the processing chamber 11, respectively. The transmitting windows are sealed by, e.g., transparent quartz glass. Further, grid-shaped detectors 20 are provided under the transmitting windows to opposedly face the windows. The amount of irradiation is detected based on electrons colliding with the detectors 20, and, then, a detection signal is inputted into the controller 14. Based on the detection signal from the detectors 20, the controller 14 controls respective outputs of the first to third electron beam sets having the first to third electron beam tubes 13A to 13C arranged in a concentric circular shape.

Further, the film reforming method of this embodiment, which employs the electron beam processor 10, has a characteristic feature in that a photoresist film layer, i.e., a film layer to be reformed, is reformed by irradiating electron beams thereon in a state where the photoresist film layer is cooled.

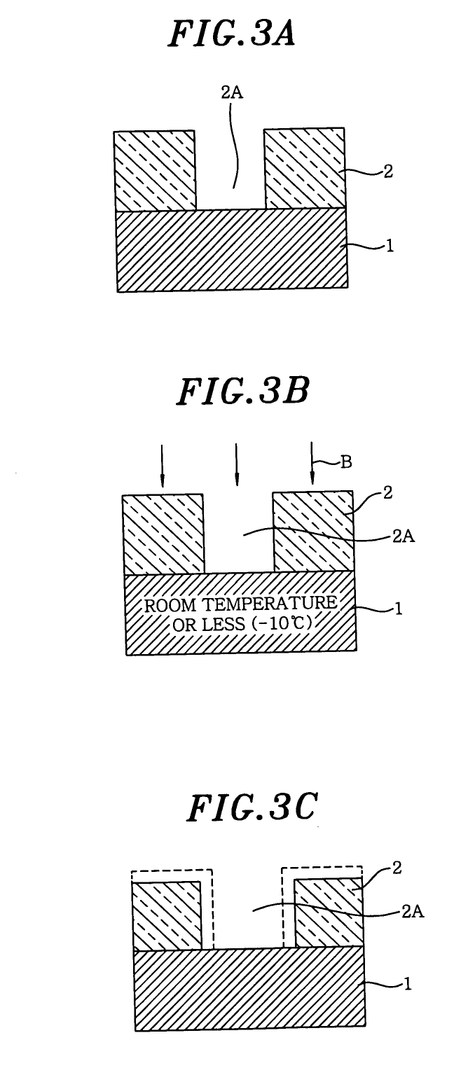

In other words, as illustrated in FIG. 3A, a film layer (e.g., SiO₂ film layer) 1 to be etched is formed on a top surface of a wafer (not shown) and, further, the photoresist film layer 2 made of an ArF photoresist material is formed on the SiO₂ film layer 1 by, e.g., a spin coating method. Further, as illustrated in the same drawing, the resist pattern 2A is formed by an ArF excimer laser in a lithography process. As for the ArF photoresist material, an organic material containing, e.g., alicyclic acrylate resin and/or aicyclic methacrylate resin or the like is used.

By irradiating electron beams on the photoresist film layer in a cooled state, the photoresist film layer can be cured while suppressing any changes in composition caused by a secession of CO gas or carbon compound containing C and H, thereby enabling to achieve a high-density cured photoresist film layer. Accordingly, it is possible to suppress CD changes in a resist pattern opening. Moreover, the carbon compound seceded by the irradiation of the electron beams is re-adhered to a sidewall of the cooled photoresist film layer in the resist pattern opening, so that a surface to which the carbon compound is adhered can be cured to serve as a protective film during an etching process. A cooling temperature of the photoresist film layer is preferably lower than 0° C. and, more preferably, ranges from 0° C. to −10° C. If the cooling temperature becomes higher than 0° C., the photoresist film layer is insufficiently cooled. Further, it is difficult to suppress a heat generation caused by irradiating the electron beams on the photoresist film layer, thereby increasing the temperature of the photoresist film layer. Accordingly, CO gas or the like becomes seceded, which may unpreferably increases a contraction of the photoresist film layer.

The irradiation amount of electron beams B projected to the photoresist film layer can be controlled based on a current fed to the electron beam units 13 and a radiation time. The radiation amount thereof preferably ranges from 200 μC/cm² to 2000 μC/cm². If it is smaller than 200 μC/cm², the photoresist film layer is insufficiently reformed, resulting in an undesirable curing thereof. Meanwhile, if it is greater than 2000 μC/cm², the photoresist film layer is excessively reformed, whereby it may be further contracted to unpreferably increase the CD thereof. Besides, the irradiation amount of the electron beams B projected to the photoresist film layer is influenced by gas types and gas pressures in the processing chamber 11.

A depth of the photoresist film layer reformed by the electron beams B can be controlled by an acceleration voltage of the electron beam units 13. The acceleration voltage of the electron beam units 13 preferably ranges from 10 kV to 15 kV. In this case, the acceleration voltage of the electron beams B projected to the photoresist film layer is controlled to range from 1 kV to 10 kV. In addition, the depth of the photoresist film layer reformed by the electron beams B projected thereon is influenced by gas types and gas pressures in the processing chamber 11.

A wafer W having the resist pattern shown in FIG. 3A is processed by using the electron beam processor 10 as follows. When the wafer W is transferred to the electron beam processor 10 via an arm of a transferring mechanism (not shown), the gate valve 17 is opened. Next, the arm of the transferring mechanism transfers the wafer W into the processing chamber 11 through the loading/unloading port 11A and then delivers the wafer W on the mounting table 12 prepared in the processing chamber 11. Thereafter, the arm of the transfer mechanism is retreated from the processing chamber 11 and, then, the gate valve 17 is closed, thereby maintaining an inner space of the processing chamber in a sealed state. In the mean time, the mounting table 12 is elevated via the elevation mechanism 15, thereby maintaining a predetermined distance between the wafer W and the electron beam units 13.

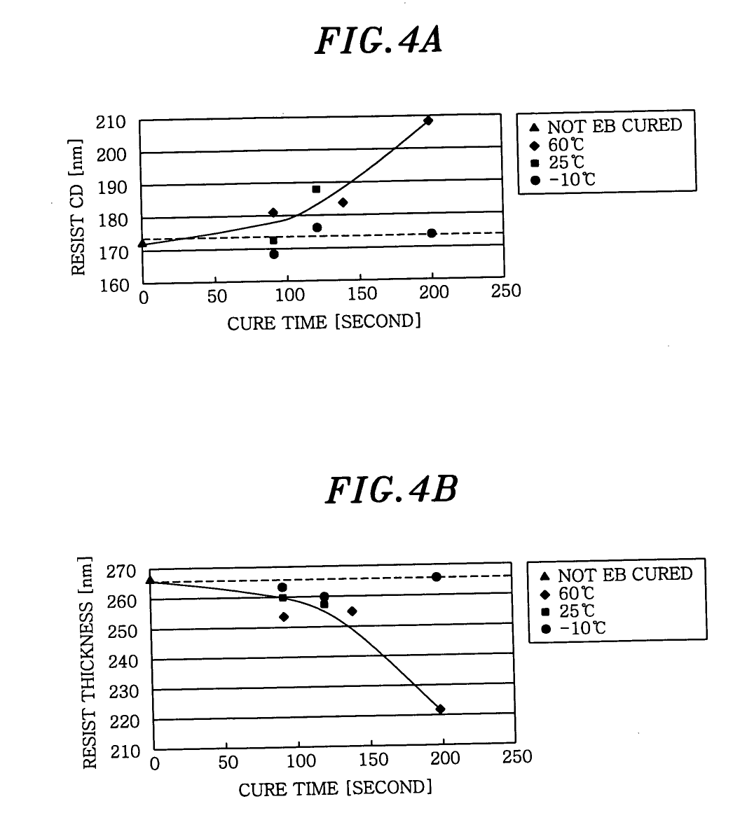

Next, under the control of the controller 14, air in the processing chamber 11 is exhausted through an exhaust unit and, at the same time, rare gas (e.g., Ar gas) is supplied from a gas supply source into the processing chamber 11, thereby substituting Ar gas for air in the processing chamber 11. Further, in a state where the wafer W is cooled by the cooling unit 12A in the processing chamber 11, the electron beams B are irradiated as illustrated in FIG. 3B while outputs of the first to third electron beam tubes 13A to 13C of the electron beam units 13 being controlled to be same. Then, the EB curing process is performed on the photoresist film layer 2 on a surface of the wafer W under following conditions, thereby curing the photoresist film layer 2. At this time, a temperature of the photoresist film layer 2 is set to be −10° C., as will be shown in following conditions. A relationship between an EB cure time and a CD of an opening of the resist pattern 2A of the photoresist film layer 2 is indicated by ● in FIG. 4A. Further, a relationship between an EB cure time and a film thickness of the photoresist film layer 2 is indicated by ● in FIG. 4B. Here and hereinafter, CD indicates an upper value of the opening.

In order to find what effect a cooling temperature has on a reforming of the photoresist film layer 2, an EB curing process was performed while setting a temperature of the photoresist film layer 2 at 25° C. and 60° C., and the results thereof are respectively shown in FIGS. 4A and 4B. Further, a CD and a film thickness of the photoresist film layer that was not subjected to the EB curing process are also shown in FIGS. 4A and 4B. Furthermore, in FIGS. 4A and 4B, ▪, ♦ and ▴ indicate states when the film layer being respectively EB cured at 25° C. and 60° C., and a state when it was not EB cured, respectively.

[Process Conditions]
Photoresist film layer: aicyclic methacrylate resin-based ArF resist material
Average film thickness: 300 nm
He gas pressure: 1 Torr
Wafer temperature: −10° C.
Ar gas flow rate: 3 L/min in a standard state
Distance between electron beam tube and wafer: 100 mm
Electron beam tube
   applied voltage: 19 kV
   tube current: 250 μA/each

From the results shown in FIGS. 4A and 4B, the CD and the film thickness of the photoresist film layer treated at −10° C. are found to show modest changes in comparison with the untreated state. Moreover, the CD and the film thickness of the photoresist film layer treated at 25° C. are found to show more changes in comparison with those shown in the treatment at −10° C. Meanwhile, the photoresist film layer treated at 60° C. is found to show the same results as those treated at 25° C. until the EB cure time reaches 150 seconds. However, after the EB cure time had elapsed 150 seconds, the CD sharply increased and the film thickness became thin. Accordingly, in case the photoresist film layer is EB cured, it is preferable to perform a cooling process in a temperature range below 0° C. In such case, as shown in FIG. 3C, the changes in the CD and the film thickness (contraction of the photoresist film layer) can be remarkably suppressed in comparison with a conventional case. Further, when it is cooled to about room temperature, the CD and the film thickness are slightly changed. However, at 65° C., the CD and the film thickness are sharply changed as the EB cure time elapses.

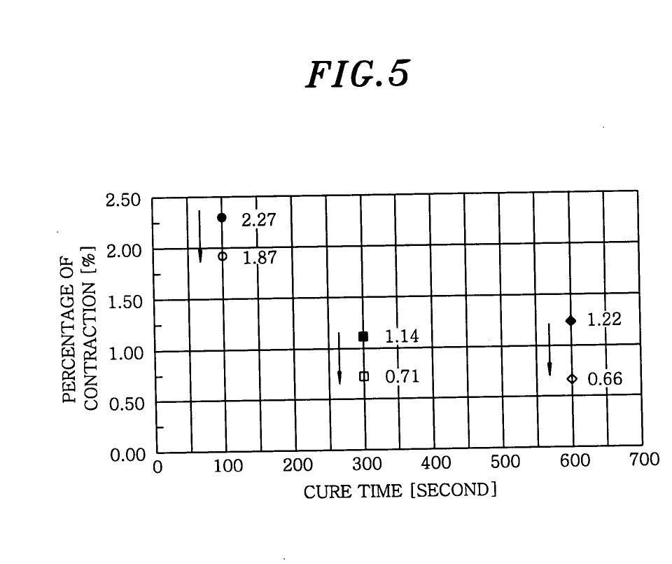

FIG. 5 provides a relationship between an EB cure time and a contraction percentage of a photoresist, which was obtained by varying EB curing process conditions. In FIG. 5, ● indicates a result obtained under the following conditions: an acceleration voltage of 19 kV, a He gas pressure of 50 Torr and a resist temperature of 25° C. Further, ◯ represents a result obtained under the same conditions as in the case indicated by ● except a resist temperature of −10° C. In addition, ▪ indicates a result obtained under the following conditions: an acceleration voltage of 13 kV, a He gas pressure of 10 Torr and a resist temperature of 25° C. □ presents a result obtained under the same conditions as in the case indicated by ▪ except a resist temperature of −10° C. Besides, ♦ indicates a result obtained under the following conditions: an acceleration voltage of 13 kV, a He gas pressure of 30 Torr and a resist temperature of 25° C. ⋄ represents a result obtained under the same conditions as in the case indicated by ♦ except a resist temperature of −10° C. In other words, the treatment has been carried out to check effects of the cooling temperature, the acceleration voltage and the He gas pressure. From the results thereof, one can deduce that when the photoresist film layer is cooled, the contraction of the photoresist film layer can be suppressed regardless of the acceleration voltage and the He gas pressure. Moreover, it can be further deduced that when the acceleration voltages are equal, the lower the He gas pressure becomes, the shorter the EB cure time becomes.

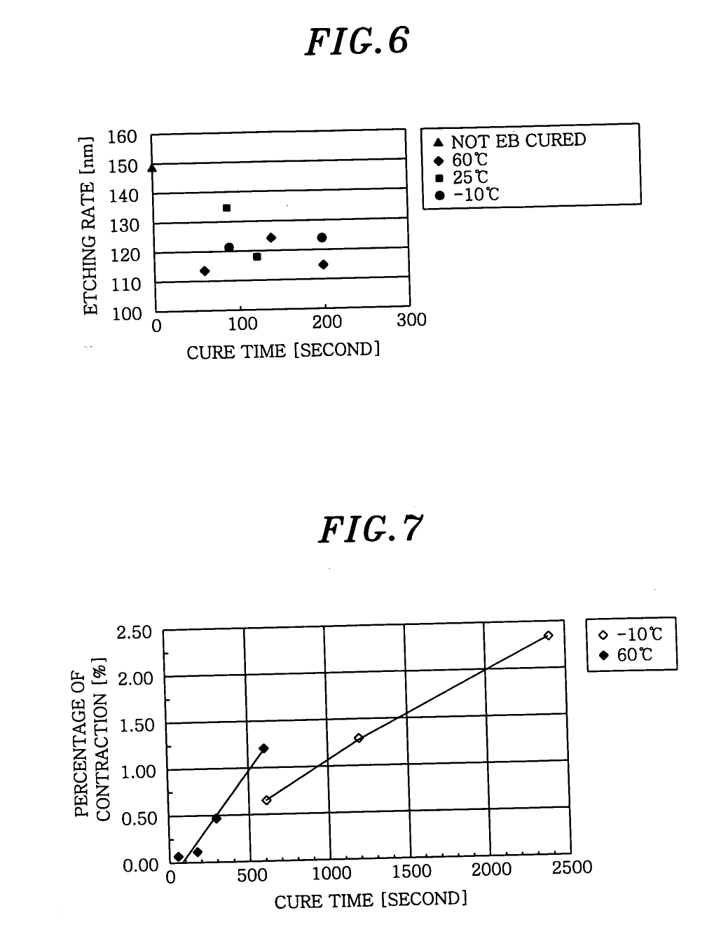

FIG. 6 depicts etching rates obtained when etching the photoresist film layer as shown in FIGS. 4A and 4B. From the result shown in FIG. 6, it can be found out that in all cases, the etching rates are decreased in comparison with that of the untreated case, and the photoresist film layer becomes cured. Moreover, a temperature of the photoresist film layer and the EB cure time are found to rarely affect the etching rate during the EB curing process. As can be seen from such result, when the treatment is carried out below 0° C., the photoresist film layer has a plasma resistance as a mask layer and, further, the CD change can be remarkably suppressed in comparison with a conventional case such that the photoresist pattern can be precisely transcribed on a film layer to be etched.

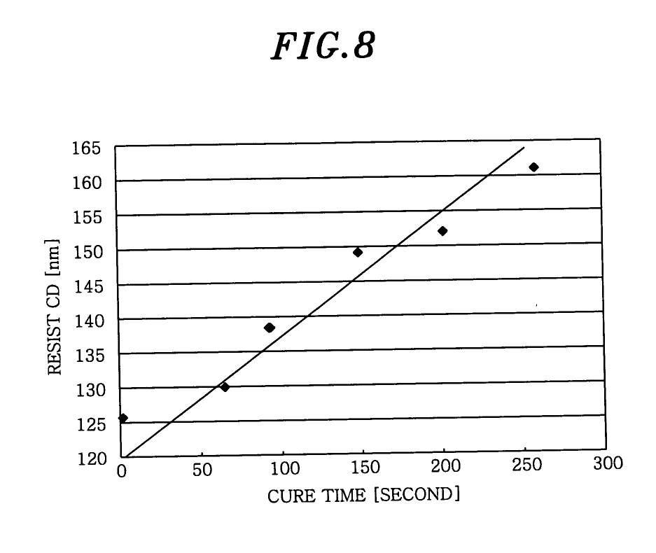

FIG. 7 illustrates a relationship between an EB cure time and a contraction percentage of the photoresist film layer, which was obtained when performing an EB curing process on the photoresist film layer under the conditions indicated by ♦ (the acceleration voltage of 13 kV, the He gas pressure of 30 Torr and the resist temperature of 25° C.) and ⋄ (the acceleration voltage of 13 kV, the He gas pressure of 30 Torr and the resist temperature of −10° C.) in FIG. 6. As can be seen from the result shown in FIG. 7, in case the EB curing process is carried out in a state where the photoresist film layer is cooled at −10° C., a changing rate (gradient) of the contraction percentage with respect to the cure time is constant and smaller than the case when performed at 60° C. Herein, a photoresist film layer having no resist pattern, i.e., a planar film of the photoresist film layer, was used.

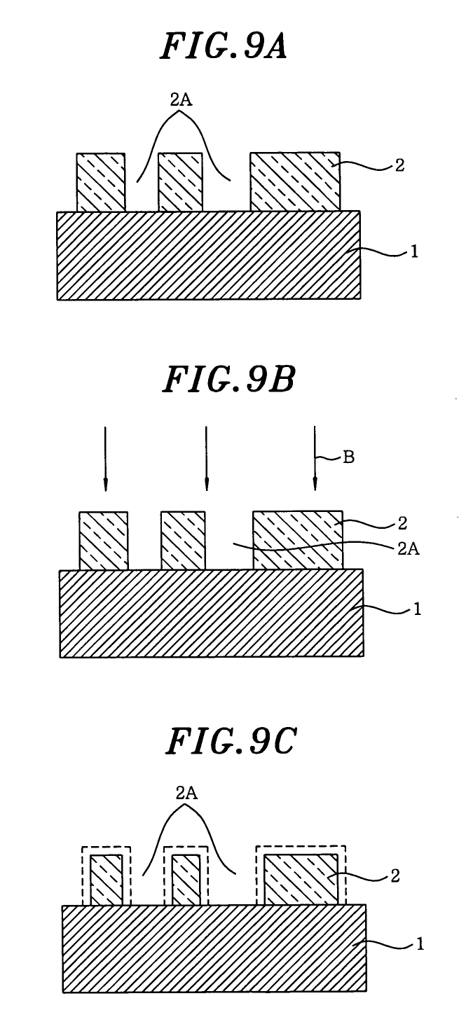

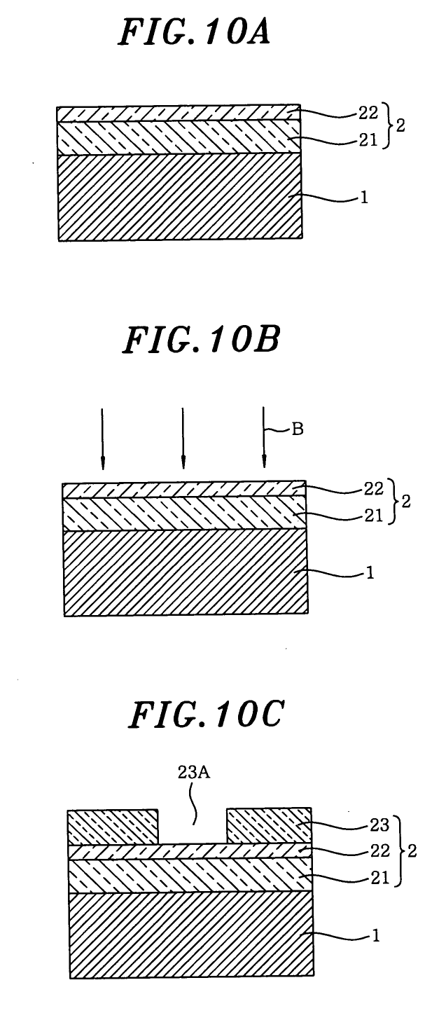

FIG. 8 provides a result obtained by examining a relationship between an EB cure time of the photoresist film layer and a CD of the resist pattern. Process conditions thereof were: the acceleration voltage of 19 kV, the electron beam tube current of 250 μA, the He gas pressure of 1 Torr and the resist temperature of 60° C. From the result shown in FIG. 8, the EB cure time is found to be in proportion to the CD of the resist pattern and, therefore, the CD can be properly controlled by controlling the EB cure time, i.e., the irradiation amount of the electron beams. Further, as illustrated in FIG. 9B, by irradiating the electron beams B on the photoresist film layer 2 having the resist pattern 2A (e.g., a wiring pattern) formed on the film layer 1 to be etched shown in FIG. 9A and further controlling the irradiation time, the wiring pattern 2A can become thin, i.e., slimmed, as indicated by a dashed line in FIG. 9C. At this time, as can be seen from the result shown in FIG. 7, in a case where the photoresist film layer is cooled to, e.g., −10° C., the wiring pattern 2A can be slimmed while the slimming amount can be more favorably controlled by controlling the cure time.

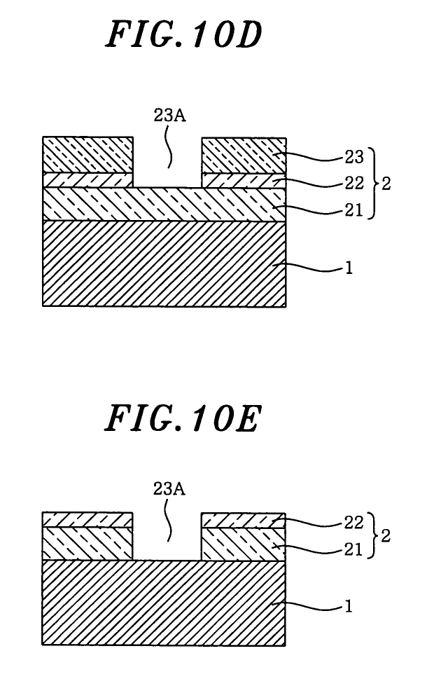

As illustrated in FIGS. 10A to 10E, the film reforming method of this embodiment can be applied to a case where the photoresist film layer 2 is a tri-layer resist. In such case, as shown in FIG. 10A, the tri-layer photoresist film layer 2 formed on a top surface of the SiO₂ film layer 1 serving as a film to be etched includes a lower layer 21 made of an organic material; an intermediate layer 22 made of an inorganic material, formed on a top surface of the lower layer 21; and an upper layer 23 made of a photoresist material, formed on a top surface of the intermediate layer 22. Such tri-layer resist is used for a multilayer wiring structure having a highly stepped surface. Such layers 21 to 23 can be formed by the spin coating method. The lower layer 21 is used for planarizing the stepped surface by way of filling stepped portions, and the intermediate layer 22 has a good etching resistance. Further, the upper layer 23 is used for forming a resist pattern by using a lithography technique.

As described in FIG. 10A, the lower and the intermediate layer 21 and 22 are formed. Then, as depicted in FIG. 10B, the lower and the intermediate layer 21 and 22 are cured by irradiating the electron beams B thereon such that each layer has a high density. Next, a top surface of the intermediate layer 22 is coated with, e.g., an ArF photoresist material, thereby forming the upper layer 23. Further, an ArF excimer laser beams are irradiated on the photoresist film layer 2 and then the photoresist is developed, thereby forming the resist pattern 23A, as illustrated in FIG. 10C. Although it is not shown, the electron beams are irradiated again in this step, thereby curing the upper layer 23. Thereafter, as shown in FIG. 10D, when the intermediate layer 22 is etched with CF-based gas by using the upper layer 23 as a mask, the resist pattern 23A of the upper layer 23 is very accurately transcribed on the intermediate layer 22. At this time, the intermediate layer 22 serves as a film layer to be etched. Next, the lower layer 21 is etched with a mixed gas of N₂ and H₂ by using the upper and the intermediate layer 23 and 22 as a mask, so that the resist pattern 23A can be very accurately transcribed on the lower layer 21. In such process, the upper layer 23 of the photoresist film layer, which is made of an organic material, is etched and removed together with the lower layer 21. When the etching is continuously carried out by using the CF-based gas, the SiO₂ film layer 1, i.e., a layer to be etched, can be etched in the shape identical to that of the resist pattern 23A of the upper layer 23 and, further, a pattern having a CD approximately same as that of the upper layer 23 can be formed.

As described above, in accordance with this embodiment, when the photoresist film layer 2 is cured by irradiating the electron beams B thereon in a state where the photoresist film layer 2 is cooled, it is possible to suppress a contraction of the photoresist film layer 2. Accordingly, a CD change of the resist pattern 2A or 23A can be remarkably suppressed such that the designed resist pattern 2A or 23A can be precisely transcribed on the SiO₂ film layer and prevent any defect on a circuit.

Furthermore, in accordance with this embodiment, when the irradiation time (the irradiation amount) of the electron beams B is controlled in a state where the photoresist film layer 2 is cooled, the slimming amount of the resist pattern 2A or 23A can be controlled and, further, it is possible to form a wiring pattern thinner than the resist pattern 2A or 23A. In other words, the pattern can be thinner than a line width formed by the ArF excimer laser.

While the invention has been shown and described with respect to the preferred embodiments, it will be understood by those skilled in the art that various changes and modification may be made without departing from the spirit and scope of the invention as defined in the following claims.

1. A film reforming method for reforming a film layer to be reformed by irradiating electron beams thereon, wherein the electron beams are irradiated in a state where the film layer is cooled.

2. The film reforming method of claim 1, wherein the film layer is cooled below 0° C.

3. The film reforming method of claim 1, wherein the film layer is an ArF resist film layer on which a pattern having a specified opening dimension is formed and a change in the specified opening dimension is suppressed by irradiating the electron beams thereon.

4. The film reforming method of claim 1, wherein the film layer is a layer to be etched that is etched through a first mask layer having a specified pattern formed thereon.

5. The film reforming method of claim 4, wherein the film layer is used as a second mask layer for etching a lower layer formed thereunder.

6. The film reforming method of claim 4, wherein the first mask layer is an ArF resist film layer.

7. The film reforming method of claim 5, wherein the second mask layer is formed by laminating an organic material layer and an inorganic material layer.

8. The film reforming method of claim 7, wherein the second mask layer is formed by a spin coating method.

9. A slimming amount controlling method for controlling a slimming amount of a resist film layer by an irradiation amount of electron beams irradiated thereon in a state where the resist film layer having a specified opening dimension is cooled.

10. The slimming amount controlling method of claim 9, wherein the resist film layer is cooled below 0° C.

11. The slimming amount controlling method of claim 9, wherein the resist film layer is an ArF resist film layer.

12. A film reforming apparatus, comprising:
a mounting unit for mounting thereon an object to be processed; and
an electron beam irradiating unit for irradiating electron beams on the object disposed on the mounting unit to thereby reform a film layer to be reformed, formed on the object,
wherein the electron beams are irradiated from the electron beam irradiating unit in a state where the film layer is cooled by a cooling unit provided in the mounting unit.

13. The film reforming apparatus of claim 12, wherein the cooling unit cools the film layer below 0° C.
